(19) United States
(12) Patent Application Publication
(10) Pub. No.: US 2008/0020048 A1
(43) Pub. Date: Jan. 24, 2008

(54) Treatment of Respiratory Disease

(76) Inventors: Susan Snape, Essex (GB); Robin Mark Bannister, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/587,718

(22) PCT Filed: May 10, 2005
(86) PCT No.: PCT/GB05/01776

§ 371 Date: Dec. 29, 2006

(30) Foreign Application Priority Data
May 10, 2004 (GB) 0410398.2

(51) Int. Cl. A61K 31/4015; A61K 9/14; A61P 11/08

(52) U.S. Cl. 424/489; 514/424

(57) ABSTRACT

Glycopyrrate or an analogue thereof is useful for the treatment of bronchospasm or as a rescue medication.

### FIELD OF THE INVENTION

This invention relates to the treatment of respiratory diseases.

### BACKGROUND OF THE INVENTION

Glycopyrrolate has been known for many years as an effective antimuscarinic agent. It has been used in several indications and been delivered by a number of different routes. It is currently used as an injectable primed to reduce secretions during anaesthesia and also as an oral product for treating gastric ulcers. One of the first descriptions of its use in airway disease was in 1984 where it was demonstrated to have a significant effect upon bronchodilation. Since then a number of studies have confirmed its potential utility.

Schroeckenstein et al., J. Allergy Clin. Immunol., 1988; 82(1): 115-119, discloses the use of glycopyrrolate in an aerosol formulation for treating asthma. A single administration of the metered-dose glycopyrrolate aerosol achieved bronchodilation over a 12 hour period.

Leckie et al., Exp. Opin. Invest. Drugs, 2000; 9(1): 3-23, is a general review of therapies for chronic obstructive pulmonary disease (COPD). Glycopyrrolate is mentioned as a possible drug treatment. However, there is no reference to its level of activity or to the duration at which it exerts its therapeutic effect.

Skorodin, Arch Intern. Med, 1993; 153: 814-828, discloses the use of glycopyrrolate in an aerosol formulation for the treatment of asthma and COPD. It is stated that, in general, the quaternary ammonium anticholinergic compounds have a duration of action of 4 to 12 hours. A dose of between 0.2 to 1.0 mg of glycopyrrolate is recommended at 6 to 12 hour intervals.

Walker et al., Chest, 1987; 91(1): 49-51, also discloses the effect of inhaled glycopyrrolate as an asthma treatment. Again, the duration of effective treatment is shown to be up to 12 hours, although up to 8 hours appears to be maximal.

WO97/39758 discloses pharmaceutical compositions for treating respiratory inflammation containing the antioxidant tyloxapol. Page 23 refers to the addition of glycopyrrolate as an additional component in solution. There is no reference to the duration of activity of the glycopyrrolate, and the proposed effective dose (200-1000 μg) is similar to that described in the prior art above.

WO01/76575 describes a pharmaceutical composition comprising an anti-muscarnic agent, for pulmonary delivery, e.g. in the treatment of asthma, COPD or cystic fibrosis. Glycopyrrolate is the preferred agent. It may be formulated with magnesium stearate.

As this composition is able to exert its therapeutic effect over a prolonged period, the patient will benefit from relief of symptoms for a longer period than with conventional anti-muscarinic treatments. Furthermore, the patient may only require a once-a-day treatment regimen, and as this will usually avoid missed treatments, better compliance is expected.

Bronchospasm is a frequent problem for those suffering from an airways disease such as asthma or COPD. Immediate relief is required. Rescue medication is required in acute bronchospasm which can be due to an acute asthma attack, exacerbation of COPD or to an allergic reaction. An acute asthma attack can be induced by, for example, exercise or environmental pollutants. The term “bronchospasm” thus includes idiopathic and non-idiopathic conditions.

### SUMMARY OF THE INVENTION

It has been found that, in addition to the benefits of glycopyrrolate therapy described in WO01/76575, various unexpected advantages have been found. Thus, for example, there is a high and immediate onset of bronchodilation. Further, it is apparent that different dosages of the drug, without side-effects, are essentially equivalent in effect. Further, problems associated with anti-muscarinics, such as tachycardia, are apparently absent. This makes the medicament particularly suitable for the treatment of bronchospasm, or as a rescue medication.

### DESCRIPTION OF THE DRAWINGS

The accompanying drawings show the results obtained in studies that illustrate the discovery underlying the present invention.

### DESCRIPTION OF THE INVENTION

The present invention utilises anti-muscarinic agents that have generally been considered to exert their pharmacological effect over a period less than 12 hours. The “pharmacological effect” relates to the ability of the agent to relieve the symptoms of the airway disorder. This may be a measure of the FEV₁ levels, which are elevated in the presence of the agent when compared to that obtained in the absence of the treatment.

Anti-muscarinics that can be used and that are structurally related to glycopyrrolate include compounds of the formula 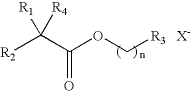
wherein n is 0, 1 or 2;

R₁ is phenyl or thiophenyl;

R₂ is H, CH₂OH, phenyl, cyclohexyl, cyclopentyl or thiophenyl;

R₃ is N⁺R₅R₆R₇ or a five or six-membered ring heterocycle containing at least one N⁺R₅R₆ group, or R₅ or R₆ is part of a ring as in

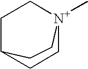

R₄is H or OH;

each of R₅, R₆, R₇ is methyl, ethyl, isopropyl or fluoroethyl; and

X⁻ is a cation, e.g. bromide or another halide, or methyl sulphate.

Examples of these drugs are benzilonium bromide, bevonium methyl sulphate, clindinium bromide, flutropium bromide, glycopyrronium bromide, heteronium bromide, hexocyclium methyl sulphate, homotropine methylbromide, ipratropium bromide, mepenzolate bromide, oxitefonium bromide, oxyphenonium bromide, oxypyrronium bromide, penthienate methobromide and pipenzolate bromide.

Further anti-muscarinics are of the formula 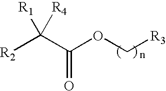
wherein n is 0, 1 or 2;

each of R₁ and R₂ is phenyl or cyclohexyl;

R₃ is NR₅R₆ or C≡CCH₂NR₃R₄ or a five or six-membered ring heterocycle containing at least one NR₅ group;

R₄is H or OH; and

each of R₅, R₆ is H, methyl, ethyl or propyl.

Examples of these drugs are benactyzine, benaprizine, dicycloverine, oxybutynin, oxyphencyclimine and piperidolate.

Glycopyrrolate is preferred, and the following description is in the context of glycopyrrolate formulations.

Glycopyrrolate has two stereogenic centres and hence exists in four isomeric forms. Each individual isomer may be delivered to optimise the efficacious effect of the drug, and reduce systemic exposure to those isomers that are responsible for systemic side-effects.

A formulation of active isomers may be used, in which the ratio of isomers is 1:1, or less than 1:1. Alternatively, the formulation of active isomers is non-racemic, or the formulation ensures that the of active isomers are delivered at different rates.

Salt forms or counterion formulations of glycopyrrolate are within the scope of the present invention, e.g. glycopyrronium bromide.

By means of the invention, glycopyrrolate can be used to treat bronchospasm, and as a rescue medication. These utilities will be evident from the evidence presented below.

Patients to be treated in accordance with the invention often suffer from complications or are undergoing other therapies. This invention has utility in treating certain patient populations, e.g. those which may have sensitivity arising from cardiovascular, ocular or mucosal complications.

Conventional formulation technology may be used to achieve desired controlled release characteristics. An important aspect is that the composition should have a duration of action greater than 12 hours, preferably more than 15 hours or 18 hours and most preferably more than 20 hours. This can be measured by techniques known to the skilled person, as shown below.

The controlled release formulations of glycopyrrolate are to be provided in a form suitable for delivery by inhalation. Devices and formulations suitable for delivery by inhalation are known to the skilled person. The composition may be prepared for delivery as an aerosol in a liquid propellant, for example for use in a pressurised metered dose inhaler (PMDI's). Propellants suitable for use in a PMDI are known to the skilled person, and include CFC-12, HFA-134a, HFA-227, HCFC-22 (difluorochloromethane), HFA-152 (difluoroethane and isobutane).

In a preferred embodiment of the invention, the compositions are in a dry powder form, for delivery using a dry powder inhaler (DPI). Dry powder inhalers are known. The dry powders for use in the inhalers will usually have a mass medium aerodynamic diameter of less than 30 μm, preferably less than 20 μm and more preferably less than 10 μm. Microparticles having aerodynamic diameters in the range of 5 to 0.5 μm will generally be deposited in the respiratory bronchioles, whereas smaller particles having aerodynamic diameters in the range of 2 to 0.05 μm are likely to be deposited in the alveoli.

The glycopyrrolate may be provided in a controlled release formulation so that fewer doses are required. Inhalers may be provided with treatment packages that supply the glycopyrrolate over an extended number of treatment days compared to packages that have a similar number of doses per pack, but from which two or three doses are required each day.

In a preferred embodiment of the invention, the glycopyrrolate is formulated with a hydrophobic material to form microparticles suitable for inhalation. The microparticles may be within the ranges specified above. Any pharmaceutically acceptable hydrophobic material may be used to formulate the microparticles, and suitable materials will be apparent to the skilled person. Preferred hydrophobic materials include solid state fatty acids such as oleic acid, lauric acid, palmitic acid, stearic acid, erucic acid, behenic acid, or derivatives (such as esters and salts) thereof. Specific examples of such materials include phosphatidylcholines, phosphatidylglycerols and other examples of natural and synthetic lung surfactants. Particularly preferred materials include metal stearates, in particular magnesium stearate, which has been approved for delivery via the lung.

The hydrophobic materials are typically resistant to immediate dissolution on administration, but are broken down over time to release the glycopyrrolate component.

The microparticles may also be formulated with additional excipients to aid delivery and release. For example, in the context of dry powder formulations, the microparticles may be formulated with additional large carrier particles which aid the flow from the dry powder inhaler into the lung. Large carrier particles are known, and include lactose particles having a mass medium aerodynamic diameter of greater than 40 μm. Alternatively, the hydrophobic microparticles may be dispersed within a carrier material. For example, the hydrophobic microparticles may be dispersed within a polysaccharide matrix, with the overall composition formulated as microparticles for direct delivery to the lung. The polysaccharide acts as a further barrier to the immediate release of the glycopyrrolate component. This may further aid the controlled release process. Suitable carrier materials will be apparent to the skilled person and include any pharmaceutically acceptable insoluble or soluble material, including polysaccharides. An example of a suitable polysaccharide is xantham gum.

The compositions may also comprise additional therapeutic agents, either as separate components, i.e. as separate microparticles, or combined with the glycopyrrolate in the microparticles. In one embodiment, a therapeutic composition comprises the microparticles according to the invention, together with microparticles consisting of the glycopyrrolate, i.e. without any hydrophobic material. This provides a composition that has a fast-acting component and a controlled-release component, and may provide effective relief quickly to a patient, together with a longer lasting effect. The fast-acting glycopyrrolate may be provided as additional microparticles, or may be dispersed, together with the hydrophobic microparticles, within a particle. For example, polysaccharide particles can be formulated with hydrophobic microparticles and fast-acting glycopyrrolate dispersed therein.

Controlled release formulations may be tested by methods known to those skilled in the art. Testing the formulations for release of glycopyrrolate in water may be used. Controlled release formulations will usually release 50% of the glycopyrrolate by dissolution in water over a period greater than 10 minutes, preferably greater than 20 minutes and most preferably greater than 30 minutes. During administration, the controlled release formulation may release the glycopyrrolate over a period greater than 12 hours, preferably 15 hours, more preferably 20 hours.

Any suitable pharmaceutically effective drug which is used for the treatment of a respiratory disease may also be co-administered with the glycopyrrolate compositions of the invention. For example, β₂-agonists, e.g. salbutamol, salmeterol and formetoral, may be formulated for co-administration with the glycopyrrolate compositions. Additional anti-muscarinic compounds may also be co-administered. For example, ipratropium (e.g. ipratropium bromide) or tiotropium may be administered. Isomers, salt forms or counterion formulations of the antimuscarinic compounds are all within the scope of the present invention. These may be in their natural form or in a controlled release formulation. The natural form is preferred.

Additional therapeutics including steroids may also be co-administered. Examples of suitable steroids include beclomethasone, dipropionate and fluticasone. Other suitable therapeutics include mucolytics, matrix metalloproteinase inhibitors, leukotrienes, antibiotics, antineoplastics, peptides, vaccines, antitussives, nicotine, PDE4 inhibitors, elastase inhibitors and sodium cromoglycate.

Combination therapy may provide the maximal effect on FEV-1 and vital capacity. Co-administration of other drugs together with the slow release glycopyrrolate may also result in less side effects compared to co-administration with the conventional glycopyrrolate formulations, as there may be less contra-indications due to the late onset of activity of the glycopyrrolate.

It is desirable that a formulation should be used, such that peak plasma levels related to systemic exposure are lower than previously, e.g. because of controlled release to give substantially constant plasma levels.

Compositions according to the invention may be produced using conventional formulation techniques. In particular, spray-drying may be used to produce the microparticles comprising the glycopyrrolate dispersed or suspended within a material that provides the controlled release properties.

The process of milling, for example, jet milling, which is also termed fluid energy milling, may also be used to formulate the therapeutic composition. The manufacture of fine particles by milling can be achieved using conventional techniques. The term “milling” is used herein to refer to any mechanical process which applies sufficient force to the particles of active material to break or grind the particles down into fine particles. A wide range of milling devices and conditions are suitable for use in the production of the compositions of the inventions. The selection of appropriate milling conditions, for example, intensity of milling and duration, to provide the required degree of force will be within the ability of the skilled person. Ball milling is a preferred method. Alternatively, a high pressure homogeniser may be used in which a fluid containing the particles is forced through a valve at high pressure producing conditions of high sheer and turbulence. Shear forces on the particles, impacts between the particles and machine surfaces or other particles, and cavitation due to acceleration of the fluid may all contribute to the fracture of the particles. Suitable homogenisers include the EmulsiFlex high pressure homogeniser, the Niro Soavi high pressure homogeniser and the Microfluidics Microfluidiser. The milling process can be used to provide the microparticles with mass median aerodynamic diameters as specified above. Milling the glycopyrrolate with a hydrophobic material is preferred, as stated above.

If it is required, the microparticles produced by the milling step can then be formulated with an additional excipient to produce particles with the hydrophobic microparticles dispersed therein. This may be achieved by a spray-drying process, e.g. co-spray-drying. In this embodiment, the hydrophobic microparticles are suspended in a solvent and co-spray-dried with a solution or suspension of the additional excipient. The spray-drying process will produce microparticles of a desired size which will comprise the hydrophobic microparticles dispersed therein. Preferred additional excipients include polysaccharides. Additional pharmaceutically effective excipients may also be used. Alternatively, the microparticles produced by the milling step can be coated with an additive using a highly intensive dry mixing method. Such methods include those termed mechanofusion or hybridisation.

The amount of the active agent to be administered will be determined by the usual factors such as the nature and severity of the disease, the condition of the patient and the potency of the agent itself. These factors can readily be determined by the skilled man. The controlled release formulation is used to sustain the bronchodilatory effect over a prolonged period and raise the FEV levels. Following initial dosing, and subsequent doses, the FEV₁ level may be maintained at a level higher than that prior to the start of the therapy. It is desirable to provide sufficient active agent so that one unit dose will enable the glycopyrrolate to exert its pharmacological effect over a period greater than 12 hours, preferably greater than 15 or 18 hours, and more preferably greater than 20 hours. The amount of glycopyrrolate released over this period will be sufficient to provide effective relief (bronchodilation) of the respiratory disease, over this period. The measurement of bronchodilation may be carried out by techniques known to the skilled person, including spirometry. This may be used to measure the FEV₁ over the administration period. It is desirable to achieve a FEV₁ value that is greater than 10% of the predicted normal value, preferably greater than 20% and most preferably greater than 30%, over the administration period. The amount of glycopyrrolate in one unit dose may be, for example, 0.02-5 mg, preferably less than 2 mg, most preferably less than or about 1 mg. Larger or smaller doses may also be provided, for example, less than 100 μg. In the context of the microparticles, the glycopyrrolate may be present in, for example, greater than 20% by weight, preferably greater than 40% by weight, and more preferably greater than 60% by weight.

The following Example illustrates the invention.

### EXAMPLE

A mixture of micronised glycopyrrolate and magnesium stearate in the ratio 75:25 by mass (total mass of approximately 1 g) was placed in a ball mill on top of 100 g of 2 mm diameter stainless steel balls. The mill volume was approximately 58.8 ml. 5 ml of cyclohexane was added to wet the mixture. The mill was sealed and secured in a Retsch S100 centrifuge. Centrification was then carried out at 500 rpm for 240 minutes in total. Small samples (approximately 5-10 mg) of wet powder were removed from the mill every 60 minutes. The samples were dried in an oven at 37° C. under vacuum.

The resultant formulation has been tested. The methodology and results are reported below.

Preliminary Study in COPD—Study Criteria

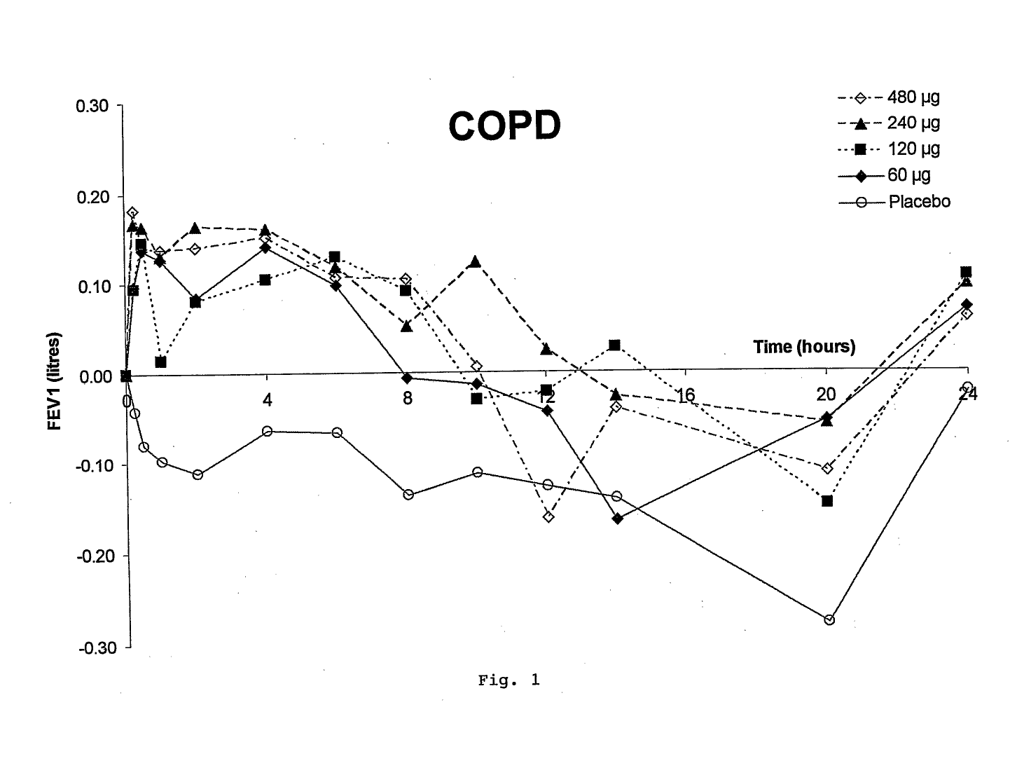

Single-dose, double blind, placebo-controlled ascending dose study
4 Treatment Days: 60→120→240→480 μg with placebo randomized into sequence
8 patients in total (1 dropout)
COPD (FEV1; FVC<70%; 45%≦FEV₁<70% predicted)
≦12% response to β₂ agonist
FEV₁ followed over 24 hours
5-7 day washout between treatments
   Results are shown in FIG. 1; see also the next table
   Preliminary Study in Asthma—Study Criteria
   Patients:
Mild—moderate asthmatics (FEV1≧55%)
Increase in FEV1≧15% and 150 ml following 80 μg Atrovent
   Part 1: Single ascending dose tolerability phase
8 patients, 2 patients per dose group to 480 μg
   Part 2: Single dose of 480 μg AD237on FEV1 compared with placebo
6 patients
responsive to Atrovent: At least 15% bronchodilation 30 mins after administration
FEV1 measured over 32 hours
   5-7 day washout between treatments

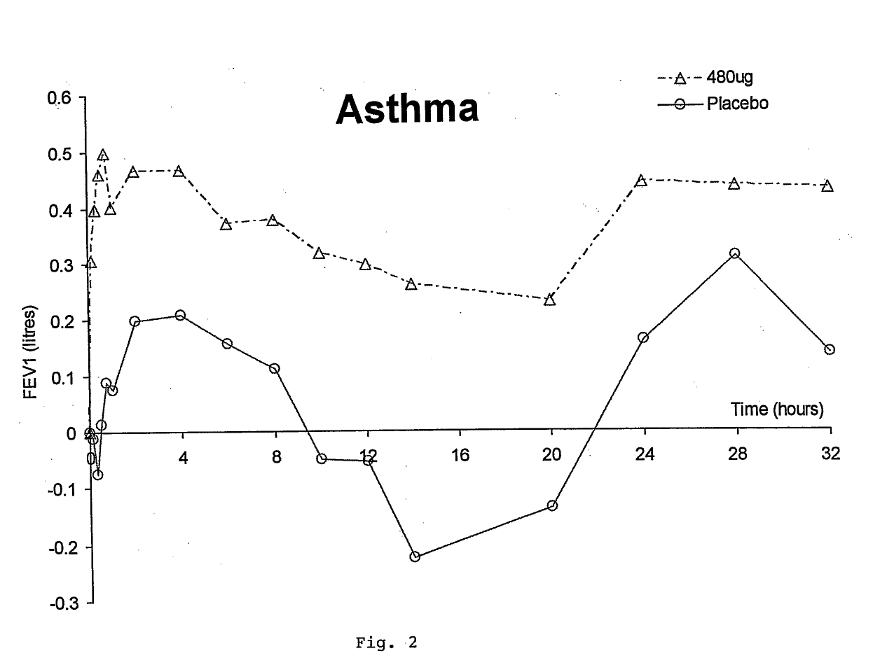

Results are shown in FIG. 2; see also Table 1.

TABLE 1

| | | Dose (μg) | | | |
|---|---|---|---|---|---|
| Change from placebo | | 60 | 120 | 240 | 480 |
| (milliliters) | | (N = 5) | (N = 6) | (N = 5) | (N = 5) |
| COPD | Peak FEV₁ | 460 | 150 | 234 | 226 |
| | Trough FEV₁ | 180 | 4 | 124 | 186 |
| Asthma | Peak FEV₁ | n/a | n/a | n/a | 430 |
| | Trough FEV₁ | n/a | n/a | n/a | 375 |

These preliminary results provided strong encouragement for proceeding into a formal
Phase IIa study.

Phase IIa COPD Dose Ranging Study

| | |
|---|---|
| Objective: | To explore the dose- and time-response of 200- |
| | 400 μg doses in patents with COPD |
| Number of centres: | 5 (UK and Germany) |
| Number of patients: | 40 |
| Design study: | Placebo-controlled, single ascending does study |
| | with placebo randomized into sequence |
| Dose: | 20, 125, 250, 400 μg AD 237 and placebo |
| Formulation: | Optimised dry powder PowderHale ® formulation |
| | (improved delivery) |
| Primary endpoint: | Weighted average change in FEV₁ (0-24 hours) |

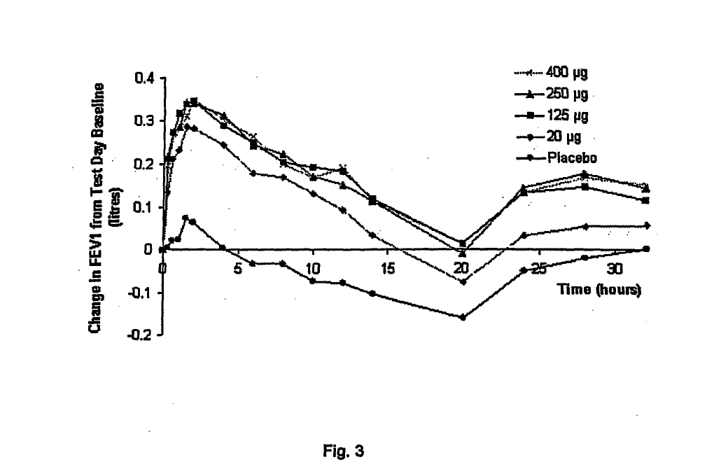

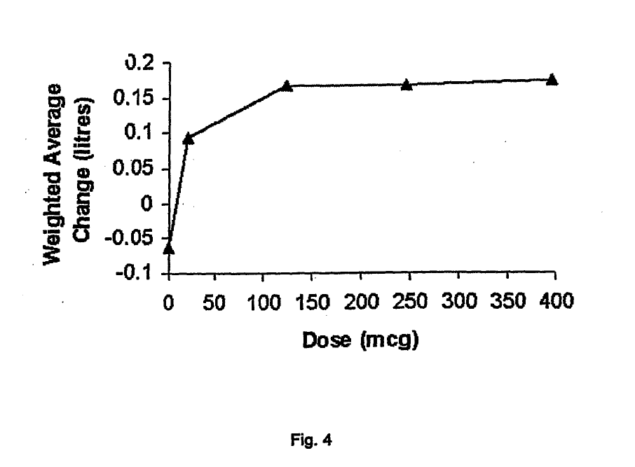

Inclusion
Diagnosis of COPD: smoking history: FEV₁ 40-80% predicted FEV1/FVC ratio <70%
Reversible airways: FEV₁ increase ≧12% and 150 ml after ipratropium
Not taking long-acting anticholinergics
   Exclusion
Susceptibility of peripheral side effects of antimuscarinics
Evidence of asthma
Unstable disease (URTI in last 6 weeks, require oxygen therapy)
Pregnancy
   Efficiency data are shown in FIG. 3; they indicate a significant effect on FEV₁ and a sustained 24-hour duration of action. Dose response is shown in FIG. 4.

A comparison was made between the 125 μg dose and 20 μg Spiriva, as described by Maesen et al, Eur. Resp. J. (1995) 8: 1506-1513. That is shown Table 2.

TABLE 2

| | Adjusted means (ml) | |
|---|---|---|
| | 125 μg | Spiriva 20 μg |
| Peak improvement in FE_{V} | 397 | 325 |
| Average improvement in FEV₁ over 24 hours | 122 | 97 |
| Trough FEV₁ | 45 | 21 |

Phase IIa Safety
No serious Adverse Events
Three severe Adverse Events
Only 1 possibly related to treatment (headache)
Most frequently reported Adverse Events were headaches (20/86 reports);
dyspnoea (5/86); sore throat (4/86) and wheeze (3/86)
Small, transient decrease in heart rate following dosing
No reports of dry mouth

1. A method for the treatment of bronchospasm, or for providing a rescue medication, wherein said method comprises administering, to a patient in need of such treatment or medication, a medicament comprising glycopyrrolate or an analogue thereof.

2. The method according to claim 1, wherein the medicament is a dry powder composition for pulmonary delivery, comprising microparticles of glycopyrrolate.

3. The method according to claim 2, wherein the microparticles have a mass median aerodynamic diameter of less than 30 μm.

4. The method according to claim 3, wherein the mass median aerodynamic diameter is 0.05 to 5 μm.

5. The method according to claim 1, wherein the medicament additionally comprises large carrier particles.

6. The method according to claim 5, wherein the large carrier particles are lactose particles having a mass median aerodynamic diameter of greater than 90 μm.

7. The method according to claim 1, wherein the medicament additionally comprises a hydrophobic material.

8. The method according to claim 7, wherein the hydrophobic material is magnesium stearate.

9. The method, according to claim 1, wherein the patient is also treated with a therapeutic agent selected from β2-agonists, steroids, mucolytics, MMP inhibitors, leukotrienes, antibiotics, antineoplastics, peptides, vaccines, antitussives, nicotine, sodium cromoglycate, PDR4 inhibitors and elastase inhibitors.

10. The method, according to claim 1, wherein the medicament is in the form of a unit dosage comprising less than 5 mg glycopyrrolate.

11. The method according to claim 10, wherein the unit dosage comprises less than 1 mg glycopyrrolate.

12. The method according to claim 1, wherein the medicament is for use as a rescue medication following an acute asthma attack.

13. The method, according to claim 1, wherein the medicament is for use as a rescue medication following exacerbation of COPD.

14. The method, according to claim 1, wherein the medicament is for use as a rescue medication following an allegic reaction.
